(19) United States
(12) Patent Application Publication
(10) Pub. No.: US 2023/0245764 A1
(43) Pub. Date: Aug. 3, 2023

(54) HEALTHCARE DATA EXCHANGE

(71) Applicant: JENNYCO, INC., San Diego, CA (US)

(72) Inventors: Michael P. Nova, Del Mar, CA (US); Jenny M. Diggles, Denver, CO (US); Linden Michael A. Leadbetter, Chesire (GB); Stephen G. Carter, Andover, MA (US)

(21) Appl. No.: 18/164,132

(22) Filed: Feb. 3, 2023

Related U.S. Application Data
(60) Provisional application No. 63/306,187, filed on Feb. 3, 2022.

(51) Int. Cl. G16H 40/20; G16H 10/60

(52) U.S. Cl. CPC G16H 40/20; G16H 10/60

(57) ABSTRACT

A system enables individuals, consumers, companies, and brands to share and access general and very specific and personalized healthcare and other data seamlessly through a blockchain service.

### CROSS-REFERENCE TO RELATED APPLICATION

The present application is a non-provisional application of, and claims priority to, U.S. Provisional Application No. 63/306,187, entitled “Healthcare Data Exchange”, filed Feb. 3, 2022, the entire disclosure of which is hereby incorporated by reference in its entirety.

### BACKGROUND

Health data of an individual is collected in a number of different manners. In some examples, an individual's health data is collected by a doctor or physician. In other examples, the individual's health data may be collected by a personal electronic device such as a wearable electronic device, a mobile telephone and the like. While various types of health data may be collected, an individual is limited in what he/she can do with that health data.

### SUMMARY

Health data associated with an individual may be valuable to that individual. Additionally, health data of an individual (and/or health data from a group of individuals) may be valuable to one or more entities. For example, a group of individuals whose health data share specific traits or outcomes may be valuable to a particular entity. As such, the present application describes the collection of health data in its many forms, the secure storage of this data, the secure and deidentified sharing of this data with groups, the personalized analysis of the individual's health data for the purpose of recommending options for an enhanced positive lifestyle or health-related issue, and the rewarding of the individual each time more data is contributed to the system or when other groups access the data of an individual as an individual or as a collective group.

In one example, the present disclosure describes a system that includes a health data exchange. Individuals and/or entities may join the health data exchange. Individuals may contribute data about themselves—including personal information, demographic information and health-related data. The data held by the data exchange may be accessible to various entities including, but not limited to, consumers, large companies, research institutes, and brands. These entities may share, access, mine and/or analyze the health data and demographic data (referred to herein as “HDM” or Human Data Markers) seamlessly through a blockchain service, which can also include a DAO (Decentralized Autonomous Organization) as managing entity. As such, an individual may provide their HDM data to the system and subsequently receive a benefit. In one example, the benefit may be a reward, monetary compensation, receipt of a blockchain token, an NFT or the like. In other examples, the benefit may be a heath recommendation, a diet recommendation, an exercise recommendation and so on. Additionally, the system described herein enables entities to gain access to cost-effective valuable HDM genomic, biomarker, physical measurements (e.g., blood pressure) and healthcare information that can better inform research and development decisions, new product creation, algorithms, and product strategy. In an example, access to an individual's data by an external group will be permitted only following the inquiry to the individual about the request and the subsequent receipt of permission to share their data from individual.

Accordingly, the present application describes a method that includes receiving, from a computing device associated with an individual, health data associated with the individual and receiving, from a computing device associated with an entity, a health data request. In an example, the health data request includes one or more parameters. A data group may be generated based, at least in part, on the health data request. The received health data is analyzed in view of the generated data group and a determination is made as to whether the received health data is candidate health data for the generated data group. Based on determining the received health data is candidate health data a determination of a benefit is made. The benefit may be based on one or more factors associated with the health data and the one or more parameters. A health data use request may be generated and provided to the computing device associated with the individual. Based on receiving confirmation of the health data request, information associated the benefit is provided to the computing device associated with the individual.

The present application also describes a system that includes at least one processing unit and a memory coupled to the at least one processing unit. The memory stores instructions that, when executed by the at least one processing unit, perform operations. In an example, the operations include receiving health data associated with an individual and receiving a health data request. In an example, the health data request is associated with an entity and includes one or more health data parameters. The received health data is analyzed in view of the health data request and a determination may be made as to whether the received health data is candidate health data for the health data request. Based on determining the received health data is candidate health data a health data use request is generated and provided to a computing device associated with the individual. Based on receiving confirmation of the health data request, the received health data is associated with the health data request.

Also described is a method that includes receiving health data associated with an individual. The method also includes receiving a health data request. In an example, the health data request is associated with an entity and includes one or more health data parameters. The received health data is analyzed in view of the health data request and a determination is made as to whether the received health data is candidate health data for the health data request. Based on determining that the received health data is candidate health data a health data use request is generated. The generated health data use request is provided to a computing device associated with the individual. Based on receiving confirmation of the health data request, the received health data is associated with the health data request.

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

### BRIEF DESCRIPTION OF THE DRAWINGS

Non-limiting and non-exhaustive examples are described with reference to the following Figures.

FIG. 1 illustrates an example system for enabling consumers and entities to share, access and query health data through an exchange system according to an example.

FIG. 2 illustrates an example method of providing a benefit to an individual in exchange for received health data according to an example.

FIG. 3 is a system diagram of a computing device according to an example.

### DETAILED DESCRIPTION

In the following detailed description, references are made to the accompanying drawings that form a part hereof, and in which are shown by way of illustrations specific embodiments or examples. These aspects may be combined, other aspects may be utilized, and structural changes may be made without departing from the present disclosure. Examples may be practiced as methods, systems, or devices. Accordingly, examples may take the form of a hardware implementation, an entirely software implementation, or an implementation combining software and hardware aspects. The following detailed description is therefore not to be taken in a limiting sense, and the scope of the present disclosure is defined by the appended claims and their equivalents.

The system described herein describes a HIPAA-compliant blockchain crypto-network having a health data (HDM) backed token (and NFT's) and a digital marketplace for data transactions (e.g., buying and selling). The transactions position owners (e.g., individuals) of health-related data and consumers of health-related data on a level HIPAA (and GDPR, HL-7) compliant playing field. For example, the system tracks the ownership of HDM (e.g., biometric data, sensor data, ethnic data, exercise data, lifestyle data, demographic data diet, living situation/location/environment, home type, whether the individual drives, walks, bikes, takes public transportation, and so on), how and when an individual should be compensated for their health-related data, as well as managing use rights and privacy of individual data records.

In examples, the system enables deployment of smart contracts (e.g., via off-chain State-channels) and applications and facilitates direct benefits (e.g., micropayments) to individuals and entities each time health data is contributed, accessed and utilized. Benefits may also be received when purchases (e.g., health data-related products and services) are made by individuals that access the system and contribute more data to a health data exchange. As will be explained in more detail below, as individuals provide health data and the health data is analyzed and used by an entity, this information (e.g., consumer data and engagements) will be recorded and validated on an immutable blockchain. For example, the blockchain may be used as both an immutable ledger and rules engine or system which sets and records various conditions for individuals that choose to lease their health data to various data pools or groups for data mining.

The system of the present disclosure enables consumers and entities to share, access and query health data seamlessly through a private exchange system. For example, the system allows an individual to own their HDM data and receive a benefit for it. Likewise, the system enables an entity (tech corporation, academic group, insurance company, food company, etc.) to gain access to valuable (and cost effective) genomic, biomarker, social, ethnic, demographic and other personal information that can better inform R&D decisions, directions, algorithms for new product creation, advertising, and product strategy based on the collected HDM data.

The system also enables or otherwise provides, a global, digital marketplace for data buying and selling transactions with an explicit base of transparency permitting the use of this data by each individual participating in the sharing of the health data. The system may also generate (e.g., by artificial intelligence (AI)) and/or provide real-time or substantially real-time product and lifestyle personalized recommendations to an individual or entity. In an example, this is achieved via blockchain state-channel application and cryptocurrency. For example, when an entity accesses the system and subsequently accesses data associated with an individual, the system provides a benefit (e.g., a micropayment, a lifestyle recommendation) to the individual and to the health data exchange.

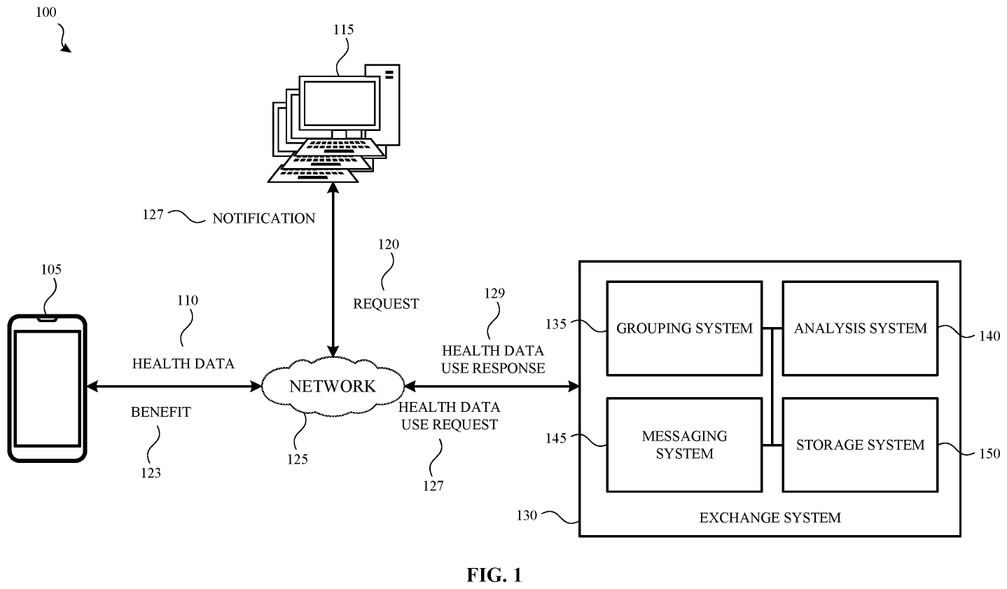

These and other examples will be shown and described in more detail with respect to FIG. 1-FIG. 3.

FIG. 1 illustrates an example system 100 for enabling consumers/individuals and entities to share, access and query health data through an exchange system 130 according to an example.

As briefly described, the system 100 may enable an individual to collect and/or provide their health data 110 to the exchange system 130. In an example, an individual may utilize a computing device 105 (e.g., a smart phone, tablet, laptop, or desktop) to actively and/or passively collect health data 110. For example, health data 110 may be collected automatically or manually by an application executing on the computing device 105. In another example, health data 110 may be collected from a third party or other mechanism (e.g., by one or more tests, doctor's appointments) and provided to the computing device 105 and/or the directly to the exchange system 130.

Once the health data is collected by the computing device 105, the health data 110 may be provided to the exchange system 130. For example, health data 110 may be provided to the exchange system 130 via a network 125. Although a network 125 is shown and described the health data 110 may be communicated or otherwise provided to the exchange system 130 via another communication channels such as, for example, Ethernet, WiFi, WAN, Bluetooth, infrared and other such communication channels.

In an example, the health data may be provided to the exchange system 130 in real time or substantially real time (e.g., as the data is collected) or periodically. In another example, the individual may choose when and/or what health data 110 is provided to the exchange system 130. Examples of health data 110 may include, but is not limited to, DNA sequence patterns, sensor data, ethnicity, biomarkers, zip code, and so on. Although specific types of data are mentioned, the exchange system 130 may collect any type of data including biological data, health/lifestyle data, social data, quantified or subjective health and well-being evaluations, demographic data and so on that is associated with an individual.

As briefly mentioned above and in another example, the health data 110 may be collected via a test kit. The test kit may be provided by an entity 115 associated with the system 100 or by a third-party. For example, the individual may receive an at-home healthcare testing kit and/or an associated data collection solution. This data collection solution may enable the individual to provide health data 110 to the exchange system 130. The health data 110 may be provided to the exchange system 130 in a number of different ways. For example, the individual may manually input their data (e.g., via a portal provided by or otherwise associated with the exchange system 130), the individual may electronically submit their data (e.g., via an application on a computing device 105) or the data may be provided to the system via a third-party (e.g., healthcare provider).

As health data 110 is collected and subsequently provided to the exchange system 130, the health data 110 may be provided to, and subsequently stored by, a storage system 150. The storage system 150 may securely store the health data 110 and/or information associated with the individual that submitted or is otherwise associated with the health data 110.

The exchange system 130 may also include an analysis system 140. The analysis system 140 may be used to determine which entities 115 have requested and/or accessed health data 110. The analysis system 140 may also determine whether an individual has given permission for one or more data groups to have access to their health data 110. In some examples, the permission may be permission for a single use of the health data 110 or for multiple uses of the health data 110. The analysis system 140 may also be used to determine a benefit 123 or a reward (e.g., a monetary value, a recommendation) for the individual that provided the health data 105.

When the benefit 123 is determined, a messaging system 145 of the exchange system 130 may automatically provide the benefit 123 to the individual (e.g., via the computing device 105 associated with the individual).

In an example, the benefit 123 is in the form of a cryptocurrency, a token, an NFT or the like. In another example, the benefit 123 is in the form of a recommendation (e.g., exercise recommendation, diet recommendation). The specifics of the benefit 123 may be based on a number of factors including, but not limited to, the amount of data provided, types of data provided, the number of times the data as accessed by an entity 115, unique features or properties associated with the data, personalization, and so on. For example, the system 100 may provide a benefit 123 to an individual for the initial uploading of health data 110, answering questions, and/or for connecting devices (e.g., computing device 105) to the exchange system 130 to enable their devices to act conduits for sharing health data 110.

The exchange system 130 may collect health data 110 (or other data) from a number of different individuals. As the health data 110 is collected or otherwise received by the exchange system 130, the analysis system 140 may aggregate, identify, analyze, and/or otherwise cause the storage system 150 to group and store similar types of data. In an example, prior to the exchange system 130 grouping and/or analyzing health data, the messaging system 145 may provide a health data use request 127 to each individual or otherwise seek permission from each individual to use their data. The heath data use request 127 may be a request for confirmation that the individual's health data 110 may be used as part of data mining group. In some examples, the health data use request 127 may also include information corresponding to the terms (e.g., duration, benefit) associated with the particular data mining group.

An individual may provide a health data use response 129 either confirming or denying the health data use request 127. In an example, the heath data use response may include a request for additional information.

For example, the exchange system 130 may include a grouping system 135 that enables entities 115 (or individuals) to provide a request 120 to the exchange system 130 to access to certain types of health data 110. In an example, the request 120 may include a request that the exchange system 130 to generate or otherwise prepare one or more data mining groups and/or a request for specific types of health data 110. For example, the data mining group may have specific terms and parameters which individuals may be required to meet, including a frequency of health data reporting, a report or accounting of how a treatment or recommendation is helping, a type of health data 110 collected and so on. The data mining group may also specify a specific reward or benefit 123 for the individual's participation. In another example, the analysis system 140 may determine the benefit 123 or otherwise provide a recommendation of the benefit to the entity 115. The benefit may be determined on past benefits 123, an amount or rarity of particular types of health data, demographic information or other factors. As such, a determined benefit or a determined value of the health data may change over time.

Individuals who agree, confirm or otherwise respond to the health data use request 127 (e.g., via a health data use response 129) and meet the specific terms and parameters of the data mining group will collect the benefit 123 when a specified term (e.g., length of time specified by an entity or other individual that sets the rules, terms, etc. of a particular data mining group) of the data mining group expires. As indicated above, a blockchain may be used as both an immutable ledger and a rules engine to set and/or conditions for individuals choosing to provide or otherwise lease their health data 110 to the various data pools or data mining groups for data mining.

As the health data 110 is collected and stored, a messaging system 145 of the exchange system 130 may provide an interface or other information that enables an entity 115 to access the health data 110, aggregate the health data 110, select health data 110 having specified characteristics and so on. A similar interface may be provided to each individual. In some examples, the inclusion of an individual's health data 110 in a group for analysis by a specific entity will proceed with the permission of the individual before the entity 115 has access to the individual's health data 110.

For example, the messaging system 145 may provide a notification to the individual that a particular entity 115 wants to access (all, some, or a portion of) the individual's health data 110. The individual may accept (or decline) to share their data with one or more entities 115 interested in the group while remaining in the group (or may opt to leave the group). In an example, the exchange system 130 may provide a cost to the entity 115 based, at least in part, on what data and/or how much data is accessed.

For example, if an entity 115 requests, collects, or otherwise analyzes 5 GB of data, the entity may be charged a particular dollar amount, token amount, or be part of a periodic service plan fee as a function of the amount and complexity of the data. As this data is accessed, the exchange system 130 tracks or otherwise identifies the owner of the data (e.g., the individual that contributed the data) and determines the associated benefit 123 to the owner.

For example, the exchange system 130 tracks and/or determines when an individual is to be provided the benefit 123 (e.g., a token). In some examples, the analysis system 140 may track a relationship between the health data 110 of an individual and a relationship between the individual's health data 110 and an entity 115. When a particular event occurs (e.g., health data is provided to a data group, the data mining session ends) information corresponding to that event is processed and an API associated the exchange system 130 will initiate transfer of the benefit 123 the computing device 105 associated with the individual.

The various costs and benefits may be transparent to the individual and the entity 115. Thus, the individual may determine or otherwise be informed as to how often their data is accessed. As such, the individual may determine whether to share more health data 110 and/or different types of health data in order to affect their benefit 123. Likewise, the entity 115 may be informed of the cost to access various types of data. Thus, both the individual and the entity 115 may feel that a fair exchange of value is occurring. However, for purposes of security and privacy, the exchange system 130 may prohibit the entity 115 from identifying the individual that provided the health data 110 and may also prohibit the individual from discovering the identity of the entity 115 that is using their data.

In order to help ensure that collected data (including data of the individual, data access patterns and frequency, monetization/benefit information) is securely stored, the storage system 150 may utilize an immutable decentralized blockchain ledger technology. This helps ensure that the collected health data 110 has value and is a movable commodity between consumers and multiple ledgers. Additionally, an individual and/or an entity 115 can interact directly with one another and/or with various aspects of the data with no intermediary following the permission from the individual.

Additionally, the exchange system 130 can provide real time or substantially real time ledger updates, append-only logs, friction free data transfer, HIPAA compliance, user consent, and privacy and security for the collected health data 110 to the individual and/or the entity 115. As indicated above, the transactions between an individual and an entity 115 may be transparent while still protecting the identity of the individual and the entity 115. The exchange system 130 may also enable automated transactions. In yet other examples, the exchange system 130 may provide transaction speed and finality of settlement between entities 115 and individuals including, but not limited to, micro-payment options.

The analysis system 140 of the exchange system 130 may also determine or otherwise learn (e.g., using one or more artificial intelligence systems) how the health data 110 collected from the various individuals is being used and/or could be used by various entities 115. As the analysis system 140 identifies this information, the messaging system 145 may provide a notification 127 that includes that information, to the entities 115. A similar notification may be provided to the individuals. However, in some examples and as indicated above, even though the individual may be notified that their data may be beneficial and/or valuable, the individual is in ultimate control of their data and may grant permission to the exchange system 130 before their data is used or otherwise provided to a data mining group.

For example, the analysis system 140 may determine that a particular entity is developing a new medicine for a particular illness. As such, the analysis system 140 may provide a notification 127 to the entity 115 that the exchange system 130 has health data 110 that would be beneficial to the development of the medicine. Additionally, the messaging system 145 of the exchange system 130 may provide a notification to one or more individuals that particular health data 110 would be beneficial to the entity that is developing that medicine.

In another example, the analysis system 140 may determine or otherwise learn that a particular entity has requested health data 110 of a particular type. The messaging system 145 may then provide a notification 127 to the entity 115 and/or to various individuals that health data 110 of a particular type is correlated with the health data 110 that has been used in the past to determine what is effective against that particular illness.

As indicated above, the exchange system 130 may receive health data from an individual. This data may be collected in a number of ways. For example, the data may be collected by the individual using an at-home lab testing service, a mobile application and/or a smart wallet, an IoT device, etc. The testing service and/or the application may provide recommendations (e.g., based on information determined by the analysis system 140) regarding the type of health data that the individual should provide to the exchange system 130. The recommendation may be based on information provided by the analysis system 140.

As indicated above, recommendations may be based on a type and/or amount of a benefit associated with the health data. For example, a particular entity may be seeking a particular type of health data. Additionally, the particular type of health data may be associated with an increased benefit 123 when compared with other data. As such, the recommendation may include a suggestion that the individual provide this particular type of health data. Once the health data is collected and provided to the exchange system, the exchange system may provide the benefit 123 to the individual.

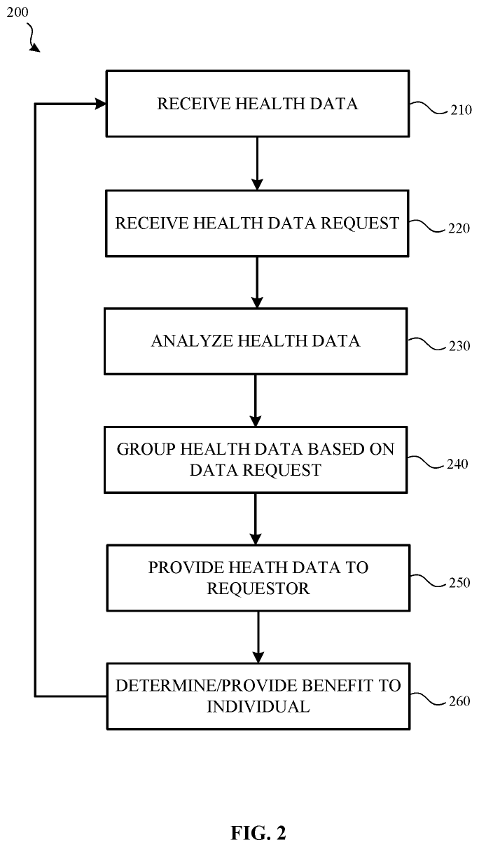

FIG. 2 illustrates an example method 200 of providing a benefit to an individual in exchange for received health data according to an example. In an example, the method 200 may be performed by one or more systems or sub-systems of the system 100 shown and described with respect to FIG. 1.

Method 200 begins when health data is received (210) from an individual. In an example and as described above, the health data may be received from a computing device associated with an individual. In other examples, the health data may be received or otherwise collected based on a test-kit, a doctor appointment, manual entry and so on.

As the health data is received, the health data may be securely stored. Additionally, personal information associated with the health data may be stored in such a way as to be compliant with HIPAA.

The method 200 also includes receiving (220) health data request from an entity. In an example, the request for health data may be received prior to, during, and/or after health data is received from the individual. In some example, the heath data request may include a request to create a data mining group such as previously described. Thus, the request for heath data may include a desired quantity of health data, a request for a particular type or types of data, desired demographic information associated with the health data, a time frame in which the data should be received, a benefit associated with the data (although a benefit may be determined by the exchange system) and so on. In an example, receipt of a health data request may trigger the generation of a permission request that is subsequently provided to an individual that owns health data identified in the request. The permission request may then be provided to an individual.

When the health data is received, an analysis system of the exchange system may analyze (230) the data. Analysis of the data may include determining the type of data that is received, how health data of different types may be correlated or otherwise related, how additional data received (e.g., data based on previously provided recommendations and/or other benefits affects health data and/or subsequent collection of data) may be associated with previously received data and so on. Likewise, the exchange system may group (240) the analyzed data based, at least in part, on the received health data request. In an example, the health data is grouped when the owner of the health data is identified as candidate health data (e.g., the health data includes information that is relevant to the data group) and the individual that owns the health data grants permission for the health data to be used. In some cases, the permission may be received prior to the group being formed, after the group has been formed or any time up until a time period associated with the group has expired.

Once the health data has been analyzed and grouped, the exchange system may provide (250) health data to the requesting entity. In some examples, the health data may be provided to the requesting entity a various times. For example, as additional health data of a particular type is provided to the exchange system, the analysis system may analyze the data and provide the additional received data of the particular type to the entity.

The exchange system may also determine and subsequently provide (260) a benefit to the individual that provided the health data. In an example, the benefit may be a recommendation. In another example, the benefit may have a monetary value. In some examples, the benefit is provided to the individual upon expiration of the data group.

As shown in FIG. 2, the method 200 (or various operations of the method 200) may be performed a number of different times. Further, each operation may be performed in any order for a number of different individuals and entities.

Additionally, health-related data may be discovered or otherwise collected in a number of different ways. For example, the system may include a data gateway system that utilizes API formats to aggregate both discrete databases and other health data aggregation providers such as popular Direct-To-Consumer (DTC) DNA and biomarker collection sites. As indicated above, any and all collected data is HIPAA compliant, consumer data is de-identified and only used with a-priori consent from the individual, and privacy is maintained by file-level and server-level encryption, along with a decentralized blockchain distributed network.

In an example, the benefit provided to the individual may be a token, a NFT, cryptocurrency or the like. This token economics (and/or the entire system) may also be managed by a DAO or Decentralized Autonomous Organization. In addition, various groups that are created based on certain types of data (e.g., blood pressure, diabetes management, mental health) can be managed by its own sub-DAO.

Based on the above, the present application provides many technical benefits including, enabling individuals to contribute healthcare data and enabling the individual to stake tokens to specific demographic liquidity pools in return for a benefit of a particular type and/or a benefit from specific pools. For example, an individual may lease their data by staking their token and providing healthcare data to the system that will, in turn, farm it or otherwise provide it to various entities when a suitable liquidity pool is identified. In another example, the individual may receive a rebate token(s) in return for purchasing lab services such as DNA or biomarker tests; for self-reported data (e.g., data captured from a wearable electronic device) such as heart rate, BP, IoT, previous genetic testing results; or for other sensor and electronic healthcare records (EHR). An individual may also be incentivized to keep their personal health data and its context (lifestyle, diet, exercise, product used, etc.) up-to-date and accurate to receive access to more liquidity pools.

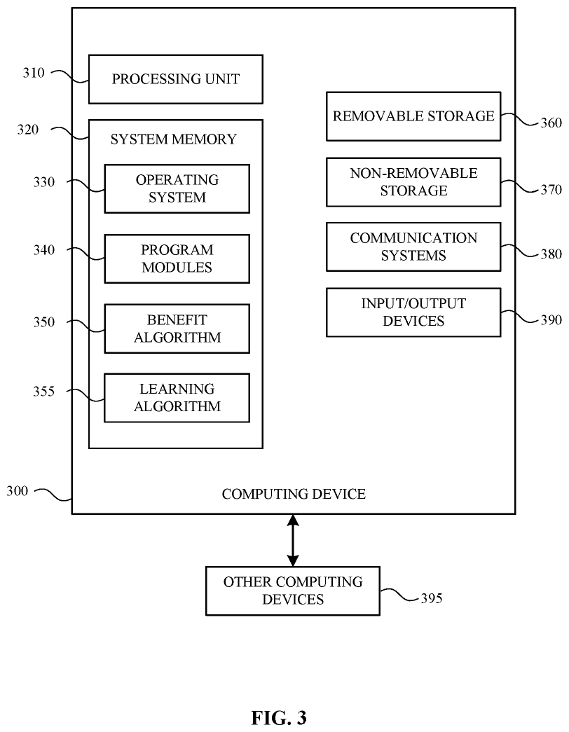

FIG. 3 is a system diagram of a computing device 300 according to an example. The computing device 300, or various components and systems of the computing device 300, may be integrated or associated with one or more systems and/or subsystems shown and described with respect to FIG. 1. For example, the computing device may be part of or otherwise integrated with the exchange system 130, the analysis system 140, the grouping system 135, the messaging system 145 and/or the storage system 150. As shown in FIG. 3, the physical components (e.g., hardware) of the computing device are illustrated and these physical components may be used to practice the various aspects of the present disclosure.

The computing device 300 may include at least one processing unit 310 and a system memory 320. The system memory 320 may include, but is not limited to, volatile storage (e.g., random access memory), non-volatile storage (e.g., read-only memory), flash memory, or any combination of such memories. The system memory 320 may also include an operating system 330 that controls the operation of the computing device 300 and one or more program modules 340. The program modules 340 may be responsible for executing and/or determining a benefits using a benefit algorithm 350. Likewise, the computing device 300 may include a learning algorithm 355 or other such artificial intelligence system that is used to monitor and/or analyze received health data. A number of different program modules and data files may be stored in the system memory 320. While executing on the processing unit 310, the program modules 340 may perform the various processes described above.

The computing device 300 may also have additional features or functionality. For example, the computing device 300 may include additional data storage devices (e.g., removable and/or non-removable storage devices) such as, for example, magnetic disks, optical disks, or tape. These additional storage devices are labeled as a removable storage 360 and a non-removable storage 370.

Examples of the disclosure may also be practiced in an electrical circuit comprising discrete electronic elements, packaged or integrated electronic chips containing logic gates, a circuit utilizing a microprocessor, or on a single chip containing electronic elements or microprocessors. For example, examples of the disclosure may be practiced via a system-on-a-chip (SOC) where each or many of the components illustrated in FIG. 3 may be integrated onto a single integrated circuit. Such a SOC device may include one or more processing units, graphics units, communications units, system virtualization units and various application functionality all of which are integrated (or “burned”) onto the chip substrate as a single integrated circuit.

When operating via a SOC, the functionality, described herein, may be operated via application-specific logic integrated with other components of the computing device 300 on the single integrated circuit (chip). The disclosure may also be practiced using other technologies capable of performing logical operations such as, for example, AND, OR, and NOT, including but not limited to mechanical, optical, fluidic, and quantum technologies.

The computing device 300 may include one or more communication systems 380 that enable the computing device 300 to communicate with other computing devices 395 such as, for example, routing engines, gateways, computing devices associated with various entities and the like. Examples of communication systems 380 include, but are not limited to, wireless communications, wired communications, cellular communications, radio frequency (RF) transmitter, receiver, and/or transceiver circuitry, a Controller Area Network (CAN) bus, a universal serial bus (USB), parallel, serial ports, etc.

The computing device 300 may also have one or more input devices and/or one or more output devices shown as input/output devices 390. These input/output devices 390 may include a keyboard, a sound or voice input device, haptic devices, a touch, force and/or swipe input device, a display, speakers, etc. The aforementioned devices are examples and others may be used.

The term computer-readable media as used herein may include computer storage media. Computer storage media may include volatile and nonvolatile, removable and non-removable media implemented in any method or technology for storage of information, such as computer readable instructions, data structures, or program modules.

The system memory 320, the removable storage 360, and the non-removable storage 370 are all computer storage media examples (e.g., memory storage). Computer storage media may include RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information and which can be accessed by the computing device 300. Any such computer storage media may be part of the computing device 300. Computer storage media does not include a carrier wave or other propagated or modulated data signal.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

The description and illustration of one or more aspects provided in this application are not intended to limit or restrict the scope of the disclosure as claimed in any way. The aspects, examples, and details provided in this application are considered sufficient to convey possession and enable others to make and use the best mode of claimed disclosure. The claimed disclosure should not be construed as being limited to any aspect, example, or detail provided in this application. Regardless of whether shown and described in combination or separately, the various features (both structural and methodological) are intended to be selectively rearranged, included or omitted to produce an embodiment with a particular set of features. Having been provided with the description and illustration of the present application, one skilled in the art may envision variations, modifications, and alternate aspects falling within the spirit of the broader aspects of the general inventive concept embodied in this application that do not depart from the broader scope of the claimed disclosure.

The term computer-readable media as used herein may include computer storage media. Computer storage media may include volatile and nonvolatile, removable and non-removable media implemented in any method or technology for storage of information, such as computer readable instructions, data structures, or program modules. Computer storage media may include RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information and which can be accessed by a computing device (e.g., host device 102 (FIG. 1)). Any such computer storage media may be part of the computing device. Computer storage media does not include a carrier wave or other propagated or modulated data signal.

Additionally, examples described herein may be discussed in the general context of computer-executable instructions residing on some form of computer-readable storage medium, such as program modules, executed by one or more computers or other devices. By way of example, and not limitation, computer-readable storage media may comprise non-transitory computer storage media and communication media. Generally, program modules include routines, programs, objects, components, data structures, etc., that perform particular tasks or implement particular abstract data types. The functionality of the program modules may be combined or distributed as desired in various examples.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

In accordance with the above, examples of the present application describe a method that includes receiving, from a computing device associated with an individual, health data associated with the individual; receiving, from a computing device associated with an entity, a health data request, the health data request including one or more parameters; generating a data group based, at least in part, on the health data request; analyzing the received health data in view of the generated data group; determining whether the received health data is candidate health data for the generated data group; based on determining the received health data is candidate health data: determining, based on one or more factors associated with the health data and the one or more parameters, a benefit associated with the health data; generating a health data use request; and providing the generated health data use request to the computing device associated with the individual; and based on receiving confirmation of the health data request, providing information associated the benefit to the computing device associated with the individual. In an example, the method also includes aggregating the health data with previously collected health data. In an example, the health data is aggregated with previously collected health data based, at least in part, on the one or more parameters associated with the health data request. In an example, the method also includes providing access to the aggregated health data to the computing device associated with the entity. In an example, the health data and the benefit is associated with a blockchain transaction. In an example, the method also includes providing information associated with an additional benefit to the computing device associated with the individual based on the individual providing additional data. In an example, the health data use request includes information corresponding to the benefit associated with the health data. In an example, the confirmation is a single-use confirmation. In an example, the confirmation is a multiple-use confirmation. In an example, the information associated the benefit is provided to the computing device associated with the individual upon expiration of the data group.

Examples of the present application also describe a system, comprising: at least one processing unit; and a memory coupled to the at least one processing unit, the memory storing instructions that, when executed by the at least one processing unit, perform operations, comprising: receiving health data associated with an individual; receiving a health data request, the health data request associated with an entity and including one or more health data parameters; analyzing the received health data in view of the health data request; determining whether the received health data is candidate health data for the health data request; based on determining the received health data is candidate health data: generating a health data use request; and providing the generated health data use request to a computing device associated with the individual; and based on receiving confirmation of the health data request, associating the received health data with the health data request. In an example, the system also includes instructions for determining, based on one or more factors associated with the health data and the one or more parameters, a benefit associated with the health data. In an example, the system also includes instructions for providing information associated the benefit to the individual. In an example, the system also includes instructions for generating a data group based, at least in part, on the health data request. In an example, information associated the benefit is provided to the individual upon expiration of the data group. In an example, the system also includes comprising instructions for aggregating the health data with additional collected health data. In an example, the health data and the benefit is associated with a blockchain transaction.

Additional examples describe a method, comprising: receiving health data associated with an individual; receiving a health data request, the health data request associated with an entity and including one or more health data parameters; analyzing the received health data in view of the health data request; determining whether the received health data is candidate health data for the health data request; based on determining the received health data is candidate health data: generating a health data use request; and providing the generated health data use request to a computing device associated with the individual; and based on receiving confirmation of the health data request, associating the received health data with the health data request. In an example, the method also includes generating a data group based, at least in part, on the health data request. In an example, the method also includes determining a benefit associated with the data group.

The description and illustration of one or more aspects provided in the present disclosure are not intended to limit or restrict the scope of the disclosure in any way. The aspects, examples, and details provided in this disclosure are considered sufficient to convey possession and enable others to make and use the best mode of claimed disclosure.

The claimed disclosure should not be construed as being limited to any aspect, example, or detail provided in this disclosure. Regardless of whether shown and described in combination or separately, the various features (both structural and methodological) are intended to be selectively rearranged, included or omitted to produce an embodiment with a particular set of features. Having been provided with the description and illustration of the present application, one skilled in the art may envision variations, modifications, and alternate aspects falling within the spirit of the broader aspects of the general inventive concept embodied in this application that do not depart from the broader scope of the claimed disclosure.

Aspects of the present disclosure have been described above with reference to schematic flowchart diagrams and/or schematic block diagrams of methods, apparatuses, systems, and computer program products according to embodiments of the disclosure. It will be understood that each block of the schematic flowchart diagrams and/or schematic block diagrams, and combinations of blocks in the schematic flowchart diagrams and/or schematic block diagrams, can be implemented by computer program instructions. These computer program instructions may be provided to a processor of a computer or other programmable data processing apparatus to produce a machine, such that the instructions, which execute via the processor or other programmable data processing apparatus, create means for implementing the functions and/or acts specified in the schematic flowchart diagrams and/or schematic block diagrams block or blocks. Additionally, it is contemplated that the flowcharts and/or aspects of the flowcharts may be combined and/or performed in any order.

References to an element herein using a designation such as “first,” “second,” and so forth does not generally limit the quantity or order of those elements. Rather, these designations may be used as a method of distinguishing between two or more elements or instances of an element. Thus, reference to first and second elements does not mean that only two elements may be used or that the first element precedes the second element. Additionally, unless otherwise stated, a set of elements may include one or more elements.

Terminology in the form of “at least one of A, B, or C” or “A, B, C, or any combination thereof” used in the description or the claims means “A or B or C or any combination of these elements.” For example, this terminology may include A, or B, or C, or A and B, or A and C, or A and B and C, or 2A, or 2B, or 2C, or 2A and B, and so on. As an additional example, “at least one of: A, B, or C” is intended to cover A, B, C, A-B, A-C, B-C, and A-B-C, as well as multiples of the same members. Likewise, “at least one of: A, B, and C” is intended to cover A, B, C, A-B, A-C, B-C, and A-B-C, as well as multiples of the same members.

Similarly, as used herein, a phrase referring to a list of items linked with “and/or” refers to any combination of the items. As an example, “A and/or B” is intended to cover A alone, B alone, or A and B together. As another example, “A, B and/or C” is intended to cover A alone, B alone, C alone, A and B together, A and C together, B and C together, or A, B, and C together.

What is claimed is:

1. A method, comprising:
receiving, from a computing device associated with an individual, health data associated with the individual;
receiving, from a computing device associated with an entity, a health data request, the health data request including one or more parameters;
generating a data group based, at least in part, on the health data request;
analyzing the received health data in view of the generated data group;
determining whether the received health data is candidate health data for the generated data group;
based on determining the received health data is candidate health data:
determining, based on one or more factors associated with the health data and the one or more parameters, a benefit associated with the health data;
generating a health data use request; and
providing the generated health data use request to the computing device associated with the individual; and
based on receiving confirmation of the health data request, providing information associated the benefit to the computing device associated with the individual.

2. The method of claim 1, further comprising aggregating the health data with previously collected health data.

3. The method of claim 2, wherein the health data is aggregated with previously collected health data based, at least in part, on the one or more parameters associated with the health data request.

4. The method of claim 2, further comprising providing access to the aggregated health data to the computing device associated with the entity.

5. The method of claim 1, wherein the health data and the benefit is associated with a blockchain transaction.

6. The method of claim 1, further comprising providing information associated with an additional benefit to the computing device associated with the individual based on the individual providing additional data.

7. The method of claim 1, wherein the health data use request includes information corresponding to the benefit associated with the health data.

8. The method of claim 1, wherein the confirmation is a single-use confirmation.

9. The method of claim 1, wherein the confirmation is a multiple-use confirmation.

10. The method of claim 1, wherein the information associated the benefit is provided to the computing device associated with the individual upon expiration of the data group.

11. A system, comprising:
at least one processing unit; and
a memory coupled to the at least one processing unit, the memory storing instructions that, when executed by the at least one processing unit, perform operations, comprising:
receiving health data associated with an individual;
receiving a health data request, the health data request associated with an entity and including one or more health data parameters;
analyzing the received health data in view of the health data request;
determining whether the received health data is candidate health data for the health data request;
based on determining the received health data is candidate health data:
generating a health data use request; and
providing the generated health data use request to a computing device associated with the individual; and
based on receiving confirmation of the health data request, associating the received health data with the health data request.

12. The system of claim 11, further comprising instructions for determining, based on one or more factors associated with the health data and the one or more parameters, a benefit associated with the health data.

13. The system of claim 12, further comprising instructions for providing information associated the benefit to the individual.

14. The system of claim 12, further comprising instructions for generating a data group based, at least in part, on the health data request.

15. The system of claim 14, wherein information associated the benefit is provided to the individual upon expiration of the data group.

16. The system of claim 11, further comprising instructions for aggregating the health data with additional collected health data.

17. The system of claim 11, wherein the health data and the benefit is associated with a blockchain transaction.

18. A method, comprising:
receiving health data associated with an individual;
receiving a health data request, the health data request associated with an entity and including one or more health data parameters;
analyzing the received health data in view of the health data request;
determining whether the received health data is candidate health data for the health data request;
based on determining the received health data is candidate health data:
generating a health data use request; and
providing the generated health data use request to a computing device associated with the individual; and
based on receiving confirmation of the health data request, associating the received health data with the health data request.

19. The method of claim 18, further comprising generating a data group based, at least in part, on the health data request.

20. The method of claim 18, further comprising determining a benefit associated with the data group.
